(19) United States
(12) Patent Application Publication
(10) Pub. No.: US 2010/0008986 A1
(43) Pub. Date: Jan. 14, 2010

(54) PHARMACEUTICAL COMPOSITIONS COMPRISING SUMATRIPTAN AND NAPROXEN

(75) Inventors: KAMAL MEHTA, BHILWARA (IN); HIDAYTULLA AGA, SATARA (IN); SANJAY KUMAR SAHU, DUBRUGARH (IN)

Correspondence Address: GLENMARK PHARMACEUTICALS INC USA, 750 CORPORATE DRIVE, MAHWAH, NJ, 07430, US

(73) Assignee: GLENMARK GENERICS, LTD., MUMBAI-400 (IN)

(21) Appl. No.: 12/501,617

(22) Filed: Jul. 13, 2009

Related U.S. Application Data
(60) Provisional application No. 61/177,678, filed on May 13, 2009.

(30) Foreign Application Priority Data
Jul. 14, 2008 (IN) 1477/MUM/2009

(51) Int. Cl. A61K 31/404; A61K 9/20; A61P 25/06

(52) U.S. Cl. 424/464; 514/415

(57) ABSTRACT

The present invention relates to the pharmaceutical composition comprising sumatriptan succinate and naproxen sodium, with a proviso that both the active components are in admixture with each other.

### PRIORITY

This application claims the benefit to Indian Provisional Application 1477/MUM/2008, filed on Jul. 14, 2008, and under 35 U.S.C. §119 to U.S. Provisional Application No. 61/177678, filed on May 13, 2009, the contents of which are incorporated by reference herein.

### BACKGROUND OF THE INVENTION

1. Technical Field

The present invention relates to the pharmaceutical composition comprising sumatriptan succinate and naproxen sodium, with a proviso that both the active components are in admixture with each other.

2. Description of the Related Art

5-HT1-like agonists and agonists acting at other 5-HT1 sites make up a group of therapeutics that may be used for the treatment of migraine headache. Representative members of this group are triptans, in particular, sumatriptan succinate (distributed under the name Imitrex® by Glaxo Wellcome, and described in U.S. Pat. No. 4,816,470).

Ergot alkaloids and related compounds are also known to have 5-HT agonist activity and have been used in migraine therapy. Among these compounds include ergotamine tartrate, ergonovine maleate, and ergoloid mesylates (e.g., dihydroergocornine, dihydroergocristine, dihydroergocryptine, and dihydroergotamine mesylate (DHE 45)). Unfortunately, it has been reported that of the 50% to 70% of patients who experience migraine symptom relief within two hours after receiving a 5-HT agonist, 30%-50% experience the recurrence of migraine symptoms within the next 24 hours. These subsequent headaches are typically termed “rebound,” “relapse,” “recurrent” or “secondary” headaches.

A variety of analgesics have also been administered to migraine patients in the form of non-steroidal anti-inflammatory drugs (NSAIDs). For example, K. M. A. Welch (New Eng. J. Med. 329:1476-1483 (1993)) sets forth the following dosages of analgesics as being useful: aspirin, 500-650 mg; acetaminophen, 500 mg; naproxen sodium, 750-825 mg; tolfenamic acid, 200-400 mg; and ibuprofen, 200 mg. However, like the 5-HT agonists, these agents, when taken alone, are rarely effective in providing complete relief symptoms and, after initial remission, migraine symptoms often return.

Recently, reports have indicated that combination therapies in which triptans are combined with NSAIDs greatly improve the relief available to migraine patients (U.S. Pat. No. 6,586,458, U.S. Pat. No. 5,872,145 and U.S. Pat. No. 6,060,499). Triptans and NSAIDs, particularly the NSAID, naproxen, have been delivered orally, for example in the form of either single layer tablets (U.S. Patent Publication 2007/0207200) or multilayer tablets (U.S. Pat. No. 7,332,183) or located in discrete zones with respect to each other wherein each zone comprises the active ingredient and optionally a carrier (U.S. Patent Publication 2007/0184109).

### SUMMARY OF THE INVENTION

The present invention provides a pharmaceutical composition comprising two active components, wherein one component is naproxen or pharmaceutically acceptable salts thereof and the other component is sumatriptan, or pharmaceutically acceptable salts thereof, with a proviso that both the active components are in admixture with each other.

The present invention provides a pharmaceutical composition comprising two active components, wherein one component is naproxen or pharmaceutically acceptable salts thereof and the other component is sumatriptan, or pharmaceutically acceptable salts thereof, with a proviso that both the active components are not at discrete zones of a pharmaceutical composition.

The present invention provides a pharmaceutical composition comprising naproxen, or pharmaceutically acceptable salts thereof and sumatriptan or pharmaceutically acceptable salts thereof, wherein the substantial quantity of Naproxen or pharmaceutically acceptable salts thereof, is present in sumatriptan component.

The present invention provides a pharmaceutical composition comprising naproxen, or pharmaceutically acceptable salts thereof and sumatriptan or pharmaceutically acceptable salts thereof, wherein the substantial quantity of Naproxen or pharmaceutically acceptable salts thereof, present in sumatriptan component is less than about 50%, preferably lees than about 30%, more preferably less than about 15%, of the total quantity of Naproxen present in composition.

The present invention provides bilayer tablet composition comprising naproxen, or pharmaceutically acceptable salts thereof and sumatriptan or pharmaceutically acceptable salts thereof, wherein the substantial quantity of Naproxen or pharmaceutically acceptable salts thereof, is present in sumatriptan component.

The present invention provides a bilayer tablet composition comprising naproxen, or pharmaceutically acceptable salts thereof and sumatriptan or pharmaceutically acceptable salts thereof, wherein the substantial quantity of Naproxen or pharmaceutically acceptable salts thereof, present in sumatriptan component is less than about 50%, preferably lees than about 30%, more preferably less than about 15%, of the total quantity of Naproxen present in composition.

In another aspect, the present invention provides a pharmaceutical composition comprising naproxen, or pharmaceutically acceptable salts thereof and sumatriptan or pharmaceutically acceptable salts thereof, wherein the substantial quantity of sumatriptan or pharmaceutically acceptable salts thereof, is present in Naproxen component.

The present invention provides a pharmaceutical composition comprising naproxen, or pharmaceutically acceptable salts thereof and sumatriptan or pharmaceutically acceptable salts thereof, wherein the substantial quantity of sumatriptan or pharmaceutically acceptable salts thereof, present in naproxen component is less than about 50%, preferably lees than about 30%, more preferably less than about 15%, of the total quantity of sumatriptan in composition.

The present invention provides bilayer tablet composition comprising naproxen, or pharmaceutically acceptable salts thereof and sumatriptan or pharmaceutically acceptable salts thereof, wherein the substantial quantity of sumatriptan or pharmaceutically acceptable salts thereof, is present in Naproxen component.

The present invention provides bilayer tablet composition comprising naproxen, or pharmaceutically acceptable salts thereof and sumatriptan or pharmaceutically acceptable salts thereof, wherein the substantial quantity of sumatriptan or pharmaceutically acceptable salts thereof, present in naproxen component is less than about 50%, preferably lees than about 30%, more preferably less than about 15%, of the total quantity of sumatriptan in composition.

The present invention further provides a pharmaceutical composition, optionally comprising carbonates, bicarbonates or equivalent materials as base components.

The present invention provides a pharmaceutical composition comprising sumaptriptan and naproxen or corresponding pharmaceutically acceptable salts thereof, wherein the sumatriptan component further comprises a carbonate and a bicarbonate or equivalent materials

The present invention further provides pharmaceutical compositions, as previously described above, wherein the sumatriptan component dissolves less than about 70% of the sumatriptan in simulated gastric fluid (SGF) within five minutes in USPII apparatus at the discriminating paddle speed of at least about 10 rpm.

The present invention provides pharmaceutical compositions, as previously described above, in capsule dosage form comprising sumatriptan adsorbed on naproxen powder.

### DETAILED DESCRIPTION OF THE INVENTION

The present invention provides a pharmaceutical composition comprising two active components, wherein one component is naproxen or pharmaceutically acceptable salts thereof and the other component is sumatriptan, or pharmaceutically acceptable salts thereof, with a proviso that both the active components are in admixture with each other.

Further, as discussed, the present invention provides a pharmaceutical composition comprising two active components, wherein one component is naproxen or pharmaceutically acceptable salts thereof and the other component is sumatriptan, or pharmaceutically acceptable salts thereof, with a proviso that both the active components are not at discrete zones of a pharmaceutical composition.

The present invention provides a pharmaceutical composition comprising naproxen or its pharmaceutically acceptable salts, preferably naproxen sodium and a sumatriptan or pharmaceutically acceptable salts thereof, preferably sumatriptan succinate with a proviso that both the active components are in admixture with each other.

As used herein, the term “stable” refers to an active compound which remains within +/−10%, preferably 6%, by weight, of the original amount, when incubated at the recited temperature for the recited amount of time in a closed container.

The pharmaceutical composition, as described above, further comprises base component/s, excipients, binders, glidants, and ancillary materials, as known and recognized by formulators skilled in the art.

The pharmaceutically acceptable excipients that may be used in the present invention include “diluents” such as lactose, lactose monohydrate, microcrystalline cellulose, dicalcium phosphate, calcium sulfate, kaolin, mannitol, sorbitol, inositol, sodium chloride, dry starch, powdered sugar and the like and mixtures thereof.

The pharmaceutically acceptable excipients that may be used in the present invention include “disintegrant” such as sodium starch glycolate, carboxymethylcellulose calcium, carboxymethylcellulose sodium (e.g. Ac-Di-Sol®, Primellose® crosscarmellose sodium), crospovidone, guar gum, magnesium aluminum silicate, methyl cellulose, microcrystalline cellulose, polacrilin potassium, powdered cellulose, pregelatinized starch, sodium alginate and starch, wherein sodium starch glycolate is most preferred.

The “binder” can be selected from one or more of hydroxypropyl methyl cellulose, polyvinylpyrrolidone, starch mucilage, pregelatinized starch, sodium alginate, alginic acid, acacia mucilage, tragacanth, carboxymethylcellulose sodium, carboxymethylcellulose calcium, ethyl cellulose, polyethylene glycol, hydroxyethyl cellulose, hydroxypropyl cellulose, methyl cellulose, polymethacrylate, carboxyvinylpolymers like Carbopols® and combinations thereof. Preferably polyvinylpyrrolidone.

Suitable “lubricants” include colloidal silicon di oxide, magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, zinc stearate and mixtures thereof. Preferably, magnesium stearate.

Suitable “glidants” include talc, colloidal silicon dioxide and combinations thereof.

Suitable “antioxidants” as stabilizers include BHA (butylated hydroxyanisole), ascorbic acid, citric acid or mixtures thereof etc. Preferably, citric acid

The base component may comprise, for example, an alkali metal or alkaline earth metal carbonate or bicarbonate, such as sodium bicarbonate, potassium bicarbonate, magnesium carbonate or calcium carbonate. The base component is preferably sodium bicarbonate. The base components may be used alone or in combination with each other. Suitably, the base component comprises from about 5% by weight to about 50% by weight, preferably about 7% by weight to about 20%, more preferably about 8% by weight to about 15% by weight, especially about 9% by weight to about 12% by weight, based on the dry weight of the layer of the dosage form.

The present invention provides a process of making a pharmaceutical composition by direct compression and/or by wet granulation and/or by dry granulation process.

The present effort is directed towards the production of combination dosage forms of sumatriptan and naproxen, wherein the individual drugs exhibit storage stability and dissolution for enhanced availability in providing pain relief to a mammal, in need thereof.

Triptans with NSAIDs are desirable therapeutic combinations, where sumatriptan is the most preferred. Sumatriptan is usually administered to patients at a dosage of between 1 mg and 300 mg with dosages of 25 mg-100 mg being preferred. Effective dosages for a variety of naproxen—tablets of 250 mg-500 mg and, for the sodium salt, tablets of 275 mg-550 mg. This information concerning tablets and dosages are provided merely as guidelines, where one of ordinary skill in the art would know and make adjustments, as required.

The present invention further provides pharmaceutical compositions, as previously described above, wherein the sumatriptan component dissolves less than about 70% of the sumatriptan in simulated gastric fluid (SGF) within five minutes in USPII apparatus at the discriminating paddle speed of at least about 10 rpm.

The present invention provides pharmaceutical compositions, as previously described above, in capsule dosage forms comprising sumatriptan adsorbed on naproxen powder.

The examples mentioned below demonstrate some illustrative procedures for preparing the pharmaceutical compositions described herein. The examples herewith illustrate the various modes that may encompass the admixture of naproxen sodium and sumatriptan succinate. In Examples 1 and 4, the active components, albeit independently granulated, are ultimately mixed intimately into a single layer. In contrast, in Example 5, the active components are mixed intimately at the onset. While in Example 2, the naproxen sodium is intimately mixed with the sumatriptan succinate, which is sprayed onto it. In Example 3, the active components, while in a bilayer tablet, the active components are still in admixture with each other, where part of the naproxen sodium is in the sumatriptan succinate layer.

The examples are provided to illustrate particular aspects of the disclosure and do not limit the scope of the present invention. The pharmaceutical compositions of the present invention can be prepared with techniques well known in the art, preferably, direct compression, dry granulation and wet granulation.

### EXAMPLES

### Example 1

### Single Layer Tablet-Independent Granulation of Naproxen Sodium and Sumatriptan Succinate

TABLE 1

| INGREDIENTS | mg/tab |
|---|---|
| INTRAGRANULAR | |
| Stage-1 | |
| Naproxen Sodium | 500.00 |
| Microcrystalline Cellulose | 50.00 |
| Crosscarmellose Sodium | 50.00 |
| Citric Acid | 50.00 |
| Povidone ® | 10.00 |
| Purified Water | qs |
| Naproxen granules coating | |
| Hydroxy propyl methyl cellulose | 12.00 |
| Purified Water | qs |
| Weight of Naproxen Sodium coated granules | 672.00 |
| Stage-II | |
| Sumatriptan Succinate | 119.00 |
| Lactose monohydrate | 50.00 |
| Crosscarmellose sodium | 50.00 |
| Polysorbate 80 | 1.00 |
| Purified Water | qs |
| Weight of Sumatriptan Succinate coated granules | 220.00 |
| EXTRAGRANULAR | |
| Microcrystalline Cellulose | 50.00 |
| Sodium Bicarbonate | 100.00 |
| Colloidal Silicon Dioxide | 10.00 |
| Talc | 10.00 |
| Magnesium Stearate | 10.00 |
| Core Tablet (Weight) | 1072.00 |
| FILM COATING | |
| Hydroxy Propyl methyl cellulose (HPMC) | 15.00 |
| Polyethylene Glycol | 4.00 |
| Talc | 3.00 |
| Titanium dioxide | 3.00 |
| Purified Water | qs |
| Film coating weight gain | 25.00 |
| Film Coated Tablet (Weight) | 1097.00 |

### Manufacturing Procedure

1) Naproxen sodium, microcrystalline cellulose, crosscarmellose sodium and citric acid are sifted through a required sieve and mixed in Rapid Mixer Granulator (RMG)

2) Povidone® is dissolved in purified water and granulated with the mixture in 1) with it in RMG.

3) The above wet mass is dried in Retsch® drier and passed through #20 mesh.

4) HPMC is dispersed in purified water.

5) Dried granules of 3) are loaded in Fluid Bed Granulator (FBE) and coated with the dispersion of 4) using a bottom spray.

6) Sumatriptan succinate, lactose monohydrate, crosscarmellose sodium are sifted through a required sieve and loaded into RMG.

7) Polysorbate® 80 is dissolved in purified water.

8) The mixture of 6) is granulated with solution of 7) in RMG.

9) The wet mass is dried in Retsch® drier and passed through #20 sieve.

10) The mixture of 5) is added with the sieved mass of 9) in a bin blender.

11) Microcrystalline cellulose, sodium bicarbonate, colloidal silicon dioxide and talc are sifted through a required sieve and mixed with the mixture of 10) in the bin blender.

12) Magnesium stearate is passed though a required sieve and used to lubricate the mixture of 11) in the bin blender.

13) The mixture of 12) is compressed to tablets using suitable punches.

14) Tablets made in 13) are coated with Opadry® solution in a Ganscoater®.

### Example 2

### Single Layer Tablet-single Layer Tablets of Naproxen Sodium and Sumatriptan in Film Coating

TABLE 2

| INGREDIENTS | mg/tab |
|---|---|
| INTRAGRANULAR | |
| Naproxen Sodium | 500.00 |
| Microcrystalline Cellulose | 100.00 |
| Citric Acid | 50.00 |
| Crosscarmellose Sodium | 60.00 |
| Povidone ® | 20.00 |
| Purified Water | qs |
| EXTRAGRANULAR | |
| Microcrystalline Cellulose | 70.00 |
| Colloidal Silicon Dioxide | 8.00 |
| Talc | 8.00 |
| Mag. Stearate | 8.00 |
| Core Tablet (Weight) | 824.00 |
| Film Coating | |
| Sumatriptan succinate | 119.00 |
| HPMC | 15.00 |
| Polyethylene Glycol | 4.00 |
| Talc | 3.00 |
| Titanium dioxide | 3.00 |
| Purified Water | qs |
| Film coated Tablet (Weight) | 968.00 |

### Manufacturing Procedure

1) Naproxen sodium, microcrystalline cellulose, crosscarmellose sodium and citric acid are sifted through 60# sieve and mixed in RMG.

2) Povidone® is dissolved in purified water and granulated with the mixture of 1) in RMG

3) The above wet mass of 3) is dried in a Retsch® drier and passed through #20 sieve.

4) Microcrystalline cellulose, colloidal silicon dioxide, talc are sifted through 60# sieve and mixed with the mixture of 3) in a bin blender.

5) Magnesium stearate is passed through a 60# sieve and used to lubricate the mixture of 4) in the bin blender.

6) The blend from 5) is compressed using suitable punches.

7) Sumatriptan succinate, HPMC, polyethylene glycol, and talc are dissolved in purified water.

8) Tablets from 6) are coated with the solution from 7) in a Ganscoater®.

### Example 3

Bilayer Tablet-Independent Granulation of Naproxen Sodium and Sumatriptan Succinate with 5% Naproxen Sodium Forming Part of Sumatriptan Layer.

TABLE 3

| INGREDIENTS | mg/tab |
|---|---|
| Stage-I | |
| Intragranular | |
| Naproxen Sodium | 475.00 |
| Microcrystalline Cellulose | 75.00 |
| Croscarmellose sodium | 50.00 |
| Povidone ® | 20.00 |
| Extra granular | |
| Colloidal silicon dioxide | 10.00 |
| Talc | 10.00 |
| Magnesium stearate | 10.00 |
| Core Tablet (Weight) | 650.00 |
| Stage-II | |
| Intragranular | |
| Sumatriptan succinate | 119.00 |
| Naproxen Sodium | 25.00 |
| Lactose monohydrate | 54.00 |
| Crosscarmellose sodium | 50.00 |
| Polysorbate ® 80 | 1.00 |
| Purified Water | Qs |
| Extra granular | |
| Sodium bicarbonate | 100.00 |
| Crosscarmellose sodium | 50.00 |
| Talc | 4.00 |
| Magnesium stearate | 4.00 |
| Core Tablet (Weight) | 407.00 |
| Bilayer Core Tablet (Weight) | 1057.00 |
| Film Coating | |
| HPMC | 15.00 |
| Polyethylene Glycol | 4.00 |
| Talc | 3.00 |
| Titanium dioxide | 3.00 |
| Purified Water | Qs |
| Film coated tablet | 1082.00 |

### Manufacturing Procedure

1) Naproxen sodium, microcrystalline cellulose and crosscarmellose sodium are sifted through 60# sieve and mixed in RMG.

2) Povidone® is dissolved in purified water and granulated with the mixture in 1) in a RMG

3) The above wet mass of 2) is dried in a Retsch® drier and passed through #20 sieve.

4) Colloidal silicon dioxide and talc are sifted through 60# sieve and mixed with mixture of 3) in a bin blender.

5) Magnesium stearate is passed through a 60# sieve and added to mixture of 4 in a bin blender.

6) Sumatriptan succinate, naproxen sodium, lactose monohydrate, crosscarmellose sodium are sifted through a 60# sieve.

7) Polysorbate® 80 is dissolved in purified water.

8) The mixture of 6) is granulated with the mixture of 7) in the RMG.

9) The wet mass of 8) is dried in a Retsch® drier and passed through #20 sieve.

10) Colloidal silicon dioxide, sodium bicarbonate and talc are sifted through a 60# sieve and mixed with granules from 9) in a bin blender.

11) Magnesium stearate is passed through a 60# sieve and added to mixture of 10) in the bin blender.

12) Using a suitable rotary tablet press (i.e. bi-layer tablet press) the final mixture of 5) naproxen sodium layer and final mixture of 11) sumatriptan layer are compressed into bilayer tablets.

13) Tablets of 12) are coated with Opadry® solution in a Ganscoater®

### Example 4

Single Layer Tablet-Separate Granulation of Naproxen Sodium and Sumatriptan Succinate and Compression into Single Layer Tablet

TABLE 4

| INGREDIENTS | mg/tab |
|---|---|
| Stage-I | |
| Intragranular | |
| Naproxen Sodium | 500.00 |
| Microcrystalline Cellulose | 50.00 |
| Crosscarmellose Sodium | 50.00 |
| Povidone ® | 20.00 |
| Polysorbate ® 80 | 1.00 |
| Purified Water | qs |
| Stage-II | |
| Intragranular | |
| Sumatriptan succinate | 119.00 |
| Lactose monohydrate | 55.00 |
| Cross carmellose Sodium | 50.00 |
| Polysorbate 80 | 1.00 |
| Purified Water | qs |
| Extra granular | |
| Sodium bicarbonate | 100.00 |
| Microcrystalline Cellulose | 40.00 |
| Croscarmellose sodium | 50.00 |
| Talc | 8.00 |
| Magnesium stearate | 8.00 |
| Core Tablet | 1052.00 |
| Final Coating | |
| Hydroxy propyl methyl cellulose | 15.00 |
| Polyethylene Glycol | 4.00 |
| Talc | 3.00 |
| Titanium dioxide | 3.00 |
| Purified Water | qs |
| Film coated Tablet | 1077.00 |

### Manufacturing Procedure

1) Naproxen sodium, microcrystalline cellulose and croscarmellose sodium are sifted through 60# sieve and mixed in RMG.

2) Povidone® and Polysorbate® 80 are dissolved in purified water and granulated with the mixture of 1) in RMG.

3) The wet mass of 2) is dried in a Retsch® drier and passed through #20 sieve.

4) Sumatriptan succinate, lactose monohydrate, and crosscarmellose sodium are sifted through 60# sieve.

5) Povidone® and Polysorbate® 80 are dissolved in purified water and granulated with the mixture of 4) in RMG.

6) The wet mass of 5) is dried in a Retsch® drier and passed through #20 sieve.

7) The granules of 3) are mixed with granules of 6) in a bin blender.

8) Microcrystalline cellulose, sodium bicarbonate, colloidal silicon dioxide and talc are sifted through 60# sieve and added to the mixture of 7) in the bin blender.

9) Magnesium stearate is passed though 60# sieve and added to 8) in the blender.

10) The blend of 9) is compressed to tablets using suitable punches.

11) Tablets of 10) are coated with Opadry® solution in a Ganscoater®.

### Example 5

### Single Layer Tablet-Both Naproxen Sodium and Sumatriptan Succinate in Single Granulation

TABLE 5

| INGREDIENTS | mg/tab |
|---|---|
| Stage-I | |
| Intragranular | |
| Naproxen Sodium | 500.00 |
| Microcrystalline Cellulose | 50.00 |
| Povidone ® | 20.00 |
| Sumatriptan succinate | 119.00 |
| Lactose monohydrate | 55.00 |
| Crosscarmellose Sodium | 60.00 |
| Polysorbate ® 80 | 2.00 |
| Purified Water | qs |
| Extra granular | |
| Sodium bicarbonate | 100.00 |
| Microcrystalline Cellulose | 42.00 |
| Croscarmellose sodium | 60.00 |
| Talc | 8.00 |
| Magnesium stearate | 8.00 |
| Core Tablet | 1024.00 |
| Final Coating | |
| Hydroxy Propyl methyl cellulose | 15.00 |
| Polyethylene Glycol | 4.00 |
| Talc | 3.00 |
| Titanium dioxide | 3.00 |
| Purified Water | qs |
| Film coated Tablet | 1049.00 |

### Manufacturing Procedure

1) Sumatriptan Succinate, naproxen sodium, lactose monohydrate, microcrystalline cellulose, croscarmellose sodium are sifted through 60# sieve and mixed in a RMG.

2) Povidone® and Polysorbate® 80 are dissolved in purified water and granulated with 1) in RMG.

3) The wet mass of 2) is dried in a Retsch drier and passed through #20 sieve.

4) Microcrystalline cellulose, sodium bicarbonate, croscarmellose sodium and talc are sifted through 60# sieve and mixed with 3) in a bin blender.

5) Magnesium stearate is passed though a required sieve and added to 4) in the blender.

6) The blend of 5) is compressed to tablets using suitable punches.

7) Tablets of 6) are coated with Opadry® solution in Ganscoater®

1. A pharmaceutical composition comprising two active components, wherein one component is naproxen or pharmaceutically acceptable salts thereof and the other component is sumatriptan, or pharmaceutically acceptable salts thereof, with a proviso that both the active components are in admixture with each other.

2. A bilayer tablet composition comprising naproxen, or pharmaceutically acceptable salts thereof and sumatriptan, pharmaceutically acceptable salts thereof, wherein the sumatriptan comprises less than about 50% by weight of naproxen or pharmaceutically acceptable salts thereof.

3. The composition of claim 2, wherein the sumatriptan comprises, less than about 25% by weight of naproxen or pharmaceutically acceptable salts thereof.

4. The composition of claim 2, wherein the sumatriptan comprises 15% by weight of naproxen or pharmaceutically acceptable salts thereof.

5. A bilayer tablet composition comprising naproxen, or pharmaceutically acceptable salts thereof and sumatriptan, or pharmaceutically acceptable salts thereof, wherein the naproxen comprises less than about 50% by weight of sumatriptan or pharmaceutically acceptable salts thereof.

6. The composition of claim 5, wherein the naproxen comprises less than about 25% by weight of sumatriptan or pharmaceutically acceptable salts thereof.

7. The composition of claim 5, wherein the naproxen comprises 15% by weight of sumatriptan or pharmaceutically acceptable salts thereof.

8. (canceled)

9. (canceled)

10. (canceled)

11. The composition as in any of claims 1, 2 or 5, optionally further comprising carbonates, bicarbonates or equivalent materials as base components.

12. The composition as in any of claims 1, 2, or 5, wherein the sumatriptan further comprises a carbonate and bicarbonate or equivalent materials.

13. The composition as in any of claims 1, 2 or 5, wherein less than about 70% of the sumatriptan is released in simulated gastric fluid (SGF) within five minutes in USPII apparatus at the discriminating paddle speed of at least about 10 rpm.
